(19) United States
(12) Patent Application Publication
(10) Pub. No.: US 2018/0196068 A1
(43) Pub. Date: Jul. 12, 2018

(54) Detection of Misfolded Amyloid Beta Protein

(71) Applicants: Board of Regents of the University of Texas System, Austin, TX (US); Amprion, Inc., San Diego, CA (US)

(72) Inventors: Claudio Soto-Jara, Friendswood, TX (US); Russell M. Lebovitz, Oakland, CA (US); Benedikt K. Vollrath, San Diego, CA (US); Mohammad Shahnawaz, Houston, TX (US)

(21) Appl. No.: 15/912,552

(22) Filed: Mar. 5, 2018

Related U.S. Application Data
(63) Continuation of application No. 14/852,471, filed on Sep. 11, 2015, now Pat. No. 9,910,049.
(60) Provisional application No. 62/049,303, filed on Sep. 11, 2014.

(51) Int. Cl. G01N 33/68

(52) U.S. Cl. CPC G01N 33/6896; G01N 2800/52; G01N 2800/2821; G01N 2333/4709

(57) ABSTRACT

Methods and kits are provided for amplifying and detecting Aβ proteins from samples, for example, from patients having Alzheimer's Disease. For example, a method for determining a presence of a soluble, misfolded Aβ protein may include contacting the sample with a monomeric, folded Aβ protein to form an incubation mixture; conducting an incubation cycle two or more times on the incubation mixture effective to form an amplified portion of misfolded Aβ protein; incubating the incubation mixture effective to cause misfolding and/or aggregation of at least a portion of the monomeric, folded Aβ protein; physically disrupting the incubation mixture effective to at least partly de-aggregate at least a portion of a misfolded Aβ aggregate present; and determining the presence of the soluble, misfolded Aβ protein in the sample by detecting at least a portion of the amplified portion of misfolded Aβ protein.

### CROSS-REFERENCE TO RELATED APPLICATIONS

This application claims priority from U.S. patent application Ser. No. 14/852,471, filed Sep. 11, 2015, and U.S. Provisional Pat. App. No. 62/049,303, filed on Sep. 11, 2014. Each of the preceding applications is entirely incorporated herein by reference.

### BACKGROUND

Protein misfolding disorders (PMDs) include Alzheimer's disease, Parkinson's disease, type 2 diabetes, Huntington's disease, amyotrophic lateral sclerosis, systemic amyloidosis, prion diseases, and the like. Misfolded aggregates of different proteins may be formed and accumulate. The misfolded aggregates may induce cellular dysfunction and tissue damage, among other effects.

For example, Alzheimer's disease (AD) is a degenerative brain disorder with no effective treatment or accurate preclinical diagnosis. Evidence to date suggests that the misfolding, aggregation, and brain deposition of the amyloid-beta protein (Aβ) may be triggering factors for AD pathology. While Aβ plaques were originally thought to be the hallmark of the disease, current research suggests that soluble Aβ oligomers may be critical synapto-toxic species causing neurodegeneration in AD. Because the brain has low regeneration capacity, early diagnosis of AD is crucial to permit intervention before irreversible neuropathological changes occur. Several lines of evidence indicate that the process of Aβ misfolding and oligomerization may begin years or decades before the onset of clinical symptoms and substantial brain damage. Current diagnosis of AD may include clinical examination complemented by imaging techniques used mainly to rule out other forms of dementia. Definitive diagnosis is done post-mortem by histological examination of the brain for the presence of amyloid plaques and neurofibrillary tangles. Still, the lack of a widely accepted early, sensitive, and objective laboratory diagnosis remains a major problem for AD care.

The present application appreciates that diagnosis of AD may be a challenging endeavor.

### SUMMARY

In one embodiment, a method for determining a presence of a soluble, misfolded Aβ protein in a sample is provided. The method may include contacting the sample with a monomeric, folded Aβ protein to form an incubation mixture. The method may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include physically disrupting the incubation mixture effective to at least partly de-aggregate at least a portion of a misfolded Aβ aggregate present. The method may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In another embodiment, a method for determining a presence of a soluble, misfolded Aβ protein in a sample is provided. The method may include contacting the sample with Thioflavin T and a monomeric, folded Aβ protein to form an incubation mixture. The method may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include shaking the incubation mixture effective to at least partly de-aggregate at least a portion of a misfolded Aβ aggregate present. The method may include determining the presence of the soluble, misfolded Aβ protein in the sample by detecting a fluorescence of the Thioflavin T corresponding to at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In one embodiment, a method for determining a presence of a soluble, misfolded Aβ protein in a sample is provided. The method may include capturing a soluble, misfolded Aβ protein from the sample to form a captured soluble, misfolded Aβ protein. The method may include contacting the captured, soluble misfolded Aβ protein with a molar excess of monomeric, folded Aβ protein to form an incubation mixture. The molar excess may be greater than an amount of Aβ protein monomer included in the captured soluble, misfolded Aβ protein. The method may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the captured soluble, misfolded Aβ protein. Each incubation cycle may include physically disrupting the incubation mixture effective to at least partly de-aggregate at least a portion of a misfolded Aβ aggregate present. The method may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The captured, soluble, misfolded Aβ protein may include one or more of: a captured, soluble, misfolded Aβ monomer and a captured, soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In another embodiment, a kit for determining a presence of a soluble, misfolded Aβ protein in a sample is provided. The kit may include one or more of a known amount of a monomeric Aβ protein and a known amount of an indicator of the soluble, misfolded Aβ protein. The kit may include instructions. The instructions may direct a user to contact the sample with one or more of the known amount of the monomeric, folded Aβ protein and the known amount of the indicator of the soluble, misfolded Aβ protein to form an incubation mixture. The instructions may direct a user to conduct an incubation cycle two or more times effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include physically disrupting the incubation mixture effective to at least partly de-aggregate at least a portion of a misfolded Aβ aggregate present. The instructions may direct a user to determine the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

The methods and kits disclosed herein for determining a presence of a soluble, misfolded Aβ protein in a sample may be effective to determine an absence of the soluble, misfolded Aβ protein in the sample.

### BRIEF DESCRIPTION OF THE DRAWINGS

The accompanying figures, which are incorporated in and constitute a part of the specification, illustrate example methods and results, and are used merely to illustrate example embodiments.

FIG. 1A shows electron micrographs taken at 0 h, 5 h, 10 h, and 24 h of incubation.

FIG. 1B is a western blot of soluble oligomeric Aβ protein aggregates.

FIG. 2A is a graph showing non-amplified amyloid formation measured by ThT fluorescence as a function of time seeded by various concentrations of synthetic soluble oligomeric Aβ protein of EXAMPLE 1.

FIG. 2B is a graph showing amplification cycle-accelerated amyloid formation measured by ThT fluorescence as a function of time seeded by various concentrations of synthetic soluble oligomeric Aβ protein of EXAMPLE 1.

FIG. 3A is a graph of amyloid formation versus time, measured as a function of ThT fluorescence labeling, showing the average kinetics of Aβ aggregation seeded by CSF from 5 representative samples from the AD, NND, and NAND groups.

FIG. 3B is a graph of the lag phase time in h for Aβ aggregation in the presence of samples from the AD, NND, and NAND groups.

FIG. 3C is a graph showing the extent of amyloid formation obtained after 180 Aβ-PMCA cycles, i.e. 90 h of incubation (P90) in the presence of CSF samples from AD, NND and NAND patients.

FIG. 4A is a plot of the true positive rate (sensitivity) as a function of the false positive rate (specificity) for different cut-off points using the lag phase values showed in FIG. 3B for AD vs. NAND.

FIG. 4B is a plot of the true positive rate (sensitivity) as a function of the false positive rate (specificity) for different cut-off points using the lag phase values showed in FIG. 3B for AD vs NND.

FIG. 4C is a plot of the true positive rate (sensitivity) as a function of the false positive rate (specificity) for different cut-off points using the lag phase values showed in FIG. 3B for AD vs All control samples.

FIG. 4D is a plot of the true positive rate (sensitivity) as a function of the false positive rate (specificity) for different cut-off points using the lag phase values showed in FIG. 3B and estimates the most reliable cut-off point for the different set of group comparisons of FIGS. 4A-4C.

FIG. 5, Table 1 shows estimations of the sensitivity, specificity and predictive value of the Aβ-PMCA test, calculated using the lag phase numbers.

FIG. 6 is a graph of the lag phase time in h for samples obtained after 300 Aβ-PMCA cycles, i.e. 150 h of incubation (P90) in the presence of CSF samples from AD and control patients.

FIG. 7A is a western blot showing results of immunodepletion using synthetically prepared Aβ oligomers spiked into human CSF.

FIG. 7B is a graph showing the kinetics of Aβ aggregation seeded by control and immunodepleted CSF samples.

FIG. 7C is a graph showing the kinetics of Aβ aggregation seeded by control and immunodepleted CSF samples, depleted only with the A11 conformational antibody.

FIG. 8A is a schematic representation of an ELISA solid phase method employed to capture Aβ oligomers from complex biological samples.

FIG. 8B is a schematic representation of a magnetic bead solid phase method employed to capture Aβ oligomers from complex biological samples.

FIG. 9, Table 2 shows the ability of specific antibodies to capture the Aβ oligomers.

FIG. 10 is a graph of amyloid formation versus time showing the acceleration of Aβ aggregation by the presence of different quantities of synthetic oligomers spiked in human plasma.

FIG. 11 is a graph showing time to reach 50% aggregation in an Aβ-PMCA assay in the presence of plasma samples from AD patients and controls.

FIG. 12 is a western blot showing the results of amplification of Aβ aggregation by cycles of incubation/sonication in the presence of distinct quantities of synthetic Aβ oligomers monitored by Western blot after protease digestion.

### DETAILED DESCRIPTION

Methods and kits are provided for the detection of misfolded proteins, specifically misfolded Aβ in a sample, including for the diagnosis of AD. This process, Protein Misfolding Cyclic Amplification (PMCA), may provide ultra-sensitive detection of misfolded aggregates through artificial acceleration and amplification of the misfolding and aggregation process in vitro. The basic concept of PMCA has been disclosed previously (Soto et al, WO 2002/04954; Estrada, et al. U.S. Pat. App. Pub. No. 20080118938, each of which is entirely incorporated herein by reference). However, prior to the present document, no patent or patent publication has enabled PCMA for the amplification and detection of misfolded Aβ in a sample, including for the diagnosis of AD. This document discloses specific examples and details which enable PMCA technology for the detection of misfolded Aβ proteins, as may be found in AD patients.

In various embodiments, methods and kits for determining a presence of a soluble, misfolded Aβ protein in a sample are provided. As described herein, methods and kits for determining a presence of a soluble, misfolded Aβ protein in a sample may be effective to determine an absence of the soluble, misfolded Aβ protein in the sample. The soluble, misfolded Aβ protein described herein may be a pathogenic protein, e.g., causing or leading to various neural pathologies associated with AD.

The methods may include contacting the sample with a monomeric, folded Aβ protein to form an incubation mixture. The methods may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include physically disrupting the incubation mixture effective to at least partly de-aggregate at least a portion of a misfolded Aβ aggregate present. The methods may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

As used herein, “Aβ” or “beta amyloid” refers to a peptide formed via sequential cleavage of the amyloid precursor protein (APP). Various Aβ isoforms may include 38-43 amino acid residues. The Aβ protein may be formed when APP is processed by β- and/or γ-secretases in any combination. The Aβ may be a constituent of amyloid plaques in brains of individuals suffering from or suspected of having AD. Various Aβ isoforms may include and are not limited to Abeta40 and Abeta42. Various Aβ peptides may be associated with neuronal damage associated with AD.

As used herein, “tau” refers to proteins are the product of alternative splicing from a single gene, e.g., MAPT (microtubule-associated protein tau) in humans. Tau proteins include to full-length and truncated forms of any of tau's isoforms. Various isoforms include, but are not limited to, the six tau isoforms known to exist in human brain tissue, which correspond to alternative splicing in exons 2, 3, and 10 of the tau gene. Three isoforms have three binding domains and the other three have four binding domains. Misfolded tau may be present in brains of individuals suffering from AD or suspected of having AD, or other tauopathies.

As used herein, “monomeric, folded Aβ protein” refers to single Aβ protein molecules in their native, nonpathogenic, folded configuration. “Soluble, misfolded Aβ protein” refers to misfolded monomers or aggregates of Aβ protein that remain in solution. Examples of soluble, misfolded Aβ protein may include any number of aggregated misfolded Aβ protein monomers so long as the misfolded Aβ protein remains soluble. For example, soluble, misfolded Aβ protein may include aggregates of between 2 and about 50 units of misfolded Aβ protein monomer. In some examples, aggregates may be referred to as oligomers or polymers. In some examples, aggregation may be referred to as oligomerization or polymerization.

Soluble, misfolded Aβ protein may aggregate or oligomerize to form insoluble aggregates and/or higher oligomers, leading to Aβ protein aggregates in the form of protofibrils, fibrils, and eventually amyloid plaques. “Seeds” or “nuclei” refer to misfolded Aβ protein or short fragmented fibrils, particularly soluble, misfolded Aβ protein, with catalytic activity for inducing further misfolding, oligomerization, and/or aggregation. Such nucleation-dependent polymerization may be characterized by a slow lag phase wherein aggregated nuclei may form, which may then catalyze rapid formation of further and/or larger aggregates. The lag phase may be minimized or removed by addition of pre-formed nuclei or seeds. In some examples, “seeds” or “nuclei” may exclude unaggregated monomers of Aβ protein. Without wishing to be bound by theory, it is believed that at least under some conditions, monomeric, misfolded Aβ protein may not be stable, and the minimum stable size of pathogenic, misfolded Aβ protein may be an aggregate of two monomer units of misfolded Aβ protein.

As used herein, “soluble” species may form a solution in biological fluids under physiological conditions, whereas “insoluble” species may be present as precipitates, fibrils, deposits, tangles, or other non-dissolved forms in such biological fluids under physiological conditions. Such biological fluids may include, for example fluids, or fluids expressed from one or more of: amniotic fluid; bile; blood; cerebrospinal fluid; cerumen; skin; exudate; feces; gastric fluid; lymph; milk; mucus, e.g. nasal secretions; mucosal membrane, e.g., nasal mucosal membrane; peritoneal fluid; plasma; pleural fluid; pus; saliva; sebum; semen; sweat; synovial fluid; tears; urine; and the like. Insoluble species may include, for example, fibrils of Aβ, αS, tau, and the like. A species that dissolves in a nonbiological fluid but not one of the aforementioned biological fluids under physiological conditions is considered insoluble, for example, the insoluble fibrils of Aβ and/or tau, and the like may be dissolved in a solution of, e.g., SDS in water, but are still considered insoluble species herein.

In some embodiments, the sample may exclude insoluble species of the misfolded proteins such as Aβ and/or tau as a precipitate, fibril, deposit, tangle, plaque, or other form that may be insoluble in one or more of the described biological fluids under physiological conditions.

For example, the sample may exclude αS and tau in fibril form. The sample may exclude misfolded Aβ, αS and/or tau proteins in insoluble form, e.g., the sample may exclude the misfolded Aβ, αS and/or tau proteins as precipitates, fibrils, deposits, tangles, plaques, or other insoluble forms, e.g., in fibril form. The methods described herein may include preparing the sample by excluding the misfolded Aβ and tau proteins in insoluble form, e.g., by excluding from the sample the misfolded Aβ and tau proteins as precipitates, fibrils, deposits, tangles, plaques, or other insoluble forms, e.g., in fibril form. The kits described herein may include instructions directing a user to prepare the sample by excluding from the sample the misfolded Aβ, αS and/or tau proteins as precipitates, fibrils, deposits, tangles, plaques, or other insoluble forms, e.g., in fibril form. The exclusion of such insoluble forms of the described misfolded proteins from the sample may be substantial or complete.

As used herein, aggregates of Aβ protein refer to non-covalent associations of protein including soluble, misfolded Aβ protein. Aggregates of Aβ protein may be “de-aggregated”, broken up, or disrupted to release smaller aggregates, e.g., soluble, misfolded Aβ protein and fragmented fibrils. The catalytic activity of a collection of misfolded Aβ protein aggregate seeds may scale, at least in part with the number of seeds in a mixture. Accordingly, disruption of aggregates of Aβ protein in a mixture to release soluble, misfolded Aβ protein and fragmented fibrils seeds may lead to an increase in catalytic activity for aggregation of monomeric Aβ protein.

As used herein, a “misfolded protein” is a protein that no longer contains all or part of the structural conformation of the protein as it exists when involved in its typical, non-pathogenic normal function within a biological system. A misfolded protein may aggregate. A misfolded protein may localize in protein aggregate. A misfolded protein may be a non-functional protein. A misfolded protein may be a pathogenic conformer of the protein. Monomeric, folded Aβ protein compositions may be provided in native, nonpathogenic confirmations without the catalytic activity for misfolding, oligomerization, and aggregation associated with seeds. Monomeric, folded Aβ protein compositions may be provided in seed-free form.

In various embodiments, methods for determining a presence of a soluble, misfolded Aβ protein in a sample are provided. The methods may include contacting the sample with Thioflavin T and a monomeric, folded Aβ protein to form an incubation mixture. The methods may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include shaking the incubation mixture effective to at least partly de-aggregate at least a portion of a misfolded Aβ aggregate present. The methods may include determining the presence of the soluble, misfolded Aβ protein in the sample by detecting a fluorescence of the Thioflavin T corresponding to at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In various embodiments, methods for determining a presence of a soluble, misfolded Aβ protein in a sample are provided. The methods may include capturing a soluble, misfolded Aβ protein from the sample to form a captured soluble, misfolded Aβ protein. The methods may include contacting the captured, misfolded Aβ protein with a molar excess of monomeric, folded Aβ protein to form an incubation mixture. The molar excess may be greater than an amount of Aβ protein monomer included in the captured soluble, misfolded Aβ protein. The methods may include conducting an incubation cycle two or more times on the incubation mixture effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the captured soluble, misfolded Aβ protein. Each incubation cycle may include physically disrupting the incubation mixture effective to at least partly de-aggregate at least a portion of a misfolded Aβ aggregate present. The methods may include determining the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The captured, soluble, misfolded Aβ protein may include one or more of: a captured, soluble, misfolded Aβ monomer and a captured, soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In some embodiments, the methods may include contacting an indicator of the soluble, misfolded protein to one or both of the incubation mixture or the detection mixture. The indicator of the soluble, misfolded Aβ protein may be characterized by an indicating state in the presence of the soluble, misfolded Aβ protein and a non-indicating state in the absence of the soluble, misfolded Aβ protein. determining the presence of the soluble, misfolded Aβ protein in the sample may include detecting the indicating state of the indicator of the soluble, misfolded Aβ protein in the detection mixture. The indicating state of the indicator and the non-indicating state of the indicator may be characterized by a difference in fluorescence, light absorption or radioactivity depending on the specific indicator. Determining the presence of the soluble, misfolded Aβ protein in the sample may include detecting the difference in these parameters.

In several embodiments, the method may include contacting a molar excess of the indicator of the soluble, misfolded Aβ protein to one or both of the incubation mixture or the detection mixture. The molar excess may be greater than a total molar amount of Aβ protein monomer included in the monomeric Aβ protein and the soluble, misfolded Aβ protein in the one or both of the incubation mixture or the detection mixture.

In various embodiments, the indicator of the soluble, misfolded Aβ protein may include one or more of: Thioflavin T, Congo Red, m-I-Stilbene, Chrysamine G, PIB, BF-227, X-34, TZDM, FDDNP, MeO-X-04, IMPY or NIAD-4, luminescent conjugated polythiophenes, a fusion with a fluorescent protein such as green fluorescent protein and yellow fluorescent protein, derivatives thereof, and the like.

In some embodiments, determining the presence of the soluble, misfolded Aβ protein in the sample may include determining an amount of the soluble misfolded Aβ protein in the sample. The amount of the soluble, misfolded Aβ protein in the sample may be determined compared to a control sample. The amount of the soluble, misfolded Aβ protein in the sample may be detected with a sensitivity of at least about one or more of: 80%, 81%, 82%, 83%, 84%, 85%, 86%, 87%, 88%, 89%, 90%, 91%, 92%, 93%, 94%, 95%, 96%, 97%, 98%, 99%, and 100%. The amount of the soluble, misfolded Aβ protein in the sample detected may be less than about one or more of: 100 nmol, 10 nmol, 1 nmol, 100 pmol, 10 pmol, 1 pmol, 100 fmol, 10 fmol, 3 fmol, 1 fmol, 100 attomol, 10 attomol, and 1 attomol. The amount of the soluble, misfolded Aβ protein in the sample may be detected in a molar ratio to monomeric Aβ protein comprised by the sample. The molar ratio may be less than about one or more of 1:100, 1:10,000, 1:100,000, and 1:1,000,000.

In various embodiments, the soluble, misfolded Aβ protein in the sample may be detected with a specificity of at least about one or more of: 80%, 81%, 82%, 83%, 84%, 85%, 86%, 87%, 88%, 89%, 90%, 91%, 92%, 93%, 94%, 95%, 96%, 97%, 98%, 99%, and 100%.

In some embodiments, the incubation mixture may include the monomeric Aβ protein in a concentration, or in a concentration range, of one or more of: between about 1 nM and about 2 mM; between about 10 nM and about 200 μM; between about 100 nM and about 20 μM; or between about 1 μM and about 10 μM; and about 2 μM.

In several embodiments, the incubation mixture may include a buffer composition. The buffer composition may be effective to prepare or maintain the pH of the incubation mixture as described herein, e.g., between pH 5 and pH 9. The buffer composition may include one or more of: Tris-HCL, PBS, MES, PIPES, MOPS, BES, TES, or HEPES, and the like. The buffer concentration may be at a total concentration of between about 1 μm and about 1M. For example, the buffer may be Tris-HCL at a concentration of 0.1 M.

In various embodiments, the incubation mixture may include a salt composition. The salt composition may be effective to increase the ionic strength of the incubation mixture. The salt composition may include one or more of: NaCl or KCl, and the like. The incubation mixture may include the salt composition at a total concentration of between about 1 μm and about 500 mM.

In several embodiments, the incubation mixture may be characterized by, prepared with, or maintained at a pH value of or a pH range of one or more of: between about 5 and about 9; between about 6 and about 8.5; between about 7 and about 8; and about 7.4.

In some embodiments, the incubation mixture may be incubated at a temperature in ° C. of about one or more of 4, 6, 8, 10, 12, 14, 16, 18, 20, 22, 24, 26, 28, 30, 32, 34, 35, 36, 37, 40, 45, 50, 55, and 60, e.g., about 22° C., or a temperature range between any two of the preceding values, for example, one or more of: between about 4° C. and about 60° C.; between about 4° C. and about 35° C.; between about 8° C. and about 50° C.; between about 12° C. and about 40° C.; between about 18° C. and about 30° C.; between about 18° C. and about 26° C.; and the like.

In several embodiments, the detecting the soluble, misfolded Aβ protein in the detection mixture may include one or more of: a Western Blot assay, a dot blot assay, an enzyme-linked immunosorbent assay (ELISA), a thioflavin T binding assay, a Congo Red binding assay, a sedimentation assay, electron microscopy, atomic force microscopy, surface plasmon resonance, spectroscopy, and the like. The ELISA may include a two-sided sandwich ELISA. The spectroscopy may include one or more of: quasi-light scattering spectroscopy, multispectral ultraviolet spectroscopy, confocal dual-color fluorescence correlation spectroscopy, Fourier-transform infrared spectroscopy, capillary electrophoresis with spectroscopic detection, electron spin resonance spectroscopy, nuclear magnetic resonance spectroscopy, Fluorescence Resonance Energy Transfer (FRET) spectroscopy, and the like.

In various embodiments, the detecting the soluble, misfolded Aβ protein in the detection mixture may include contacting the detection mixture with a protease. The soluble, misfolded Aβ protein may be detected in the detection mixture using sequence-based or anti-misfolded protein antibodies in one or more of: a Western Blot assay, a dot blot assay, and an ELISA.

In some embodiments, the method may include providing the monomeric Aβ protein in labeled form. The monomeric Aβ protein in labeled form may include one or more of: a covalently incorporated radioactive amino acid, a covalently incorporated, isotopically labeled amino acid, and a covalently incorporated fluorophore. The detecting the soluble, misfolded Aβ protein in the detection mixture may include detecting the monomeric Aβ protein in labeled form as incorporated into the amplified portion of the soluble, misfolded Aβ protein.

In several embodiments, the sample may be taken from a subject. The method may include determining or diagnosing the presence of AD in the subject according to the presence of the soluble, misfolded Aβ protein in the sample. The presence of the soluble, misfolded Aβ protein in the sample may be determined compared to a control sample taken from a control subject.

In various embodiments, the detecting may include detecting an amount of the soluble, misfolded Aβ protein in the sample. The method may include determining or diagnosing the presence of AD in the subject by comparing the amount of the soluble, misfolded Aβ protein in the sample to a predetermined threshold amount.

In several embodiments, the sample may be taken from a subject exhibiting no clinical signs of dementia according to cognitive testing. The method may include determining or diagnosing the presence of AD in the subject according to the presence of the soluble, misfolded Aβ protein in the sample.

In some embodiments, the sample may be taken from a subject exhibiting no cortex plaques or tangles according to amyloid beta contrast imaging. The method may further include determining or diagnosing the presence of AD in the subject according to the presence of the soluble, misfolded Aβ protein in the sample.

In various embodiments, the sample may be taken from a subject exhibiting clinical signs of dementia according to cognitive testing. The method may further include determining or diagnosing the presence of AD as a contributing factor to the clinical signs of dementia in the subject according to the presence of the soluble, misfolded Aβ protein in the sample.

In some embodiments, the sample may include one or more of: amniotic fluid; bile; blood; cerebrospinal fluid; cerumen; skin; exudate; feces; gastric fluid; lymph; milk; mucus, e.g. nasal secretions; mucosal membrane, e.g., nasal mucosal membrane; peritoneal fluid; plasma; pleural fluid; pus; saliva; sebum; semen; sweat; synovial fluid; tears; and urine.

In several embodiments, the method may include taking the sample from the subject. The subject may be one of a: human, mouse, rat, dog, cat, cattle, horse, deer, elk, sheep, goat, pig, or non-human primate. Non-human animals may be wild or domesticated. The subject may be one or more of: at risk of AD, having AD, and under treatment for AD, at risk of having a disease associated with dysregulation, misfolding, aggregation or disposition of Aβ, having a disease associated with dysregulation, misfolding, aggregation or disposition of Aβ, or under treatment for a disease associated with dysregulation, misfolding, aggregation or disposition of Aβ.

In various embodiments, the method may include determining or diagnosing a progression or homeostasis of AD in the subject by comparing the amount of the soluble, misfolded Aβ protein in the sample to an amount of the soluble, misfolded Aβ protein in a comparison sample taken from the subject at a different time compared to the sample.

For example, several novel therapeutics that are targeting Aβ homeostasis through various mechanisms are currently under development. A PMCA assay for Aβ oligomers may be employed to determine which patients may be treated with an Aβ modulating therapy. Patients showing a change, e.g., decrease or increase, in the level of Aβ oligomers as detected by the PMCA method may be classified as “responders” to Aβ modulating therapy, and may be treated with a therapeutic reducing the levels of Aβ oligomers. Patients lacking an aberrant Aβ homeostasis may be classified as “non responders” and may not be treated. Patients who could benefit from therapies aimed at modulating Aβ homeostasis may thus be identified.

Further, for example, the amount of Aβ oligomers may be measured in samples from patients using PMCA. Patients with elevated Aβ measurements may be treated with therapeutics modulating Aβ homeostasis. Patients with normal Aβ measurements may not be treated. A response of a patient to therapies aimed at modulating Aβ homeostasis may be followed. For example, Aβ oligomer levels may be measured in a patient sample at the beginning of a therapeutic intervention. Following treatment of the patient for a clinical meaningful period of time, another patient sample may be obtained and Aβ oligomer levels may be measured. Patients who show a change in Aβ levels following therapeutic intervention may be considered to respond to the treatment. Patients who show unchanged Aβ levels may be considered non-responding. The methods may include detection of Aβ aggregates in patient samples containing components that may interfere with the PMCA reaction.

In some embodiments, the subject may be treated with an Aβ modulating therapy. The method may include comparing the amount of the soluble, misfolded Aβ protein in the sample to an amount of the soluble, misfolded Aβ protein in a comparison sample. The sample and the comparison sample may be taken from the subject at different times over a period of time under the Aβ modulating therapy. The method may include determining or diagnosing the subject is one of: responsive to the Aβ modulating therapy according to a change in the soluble, misfolded Aβ protein over the period of time, or non-responsive to the Aβ modulating therapy according to homeostasis of the soluble, misfolded Aβ protein over the period of time. The method may include treating the subject determined to be responsive to the Aβ modulating therapy with the Aβ modulating therapy. The Aβ modulating therapy may include administration of one or more of: an inhibitor of BACE1 (beta-secretase 1); an inhibitor of y-secretase; and a modulator of Aβ homeostasis, e.g., an immunotherapeutic modulator of Aβ homeostasis. The Aβ modulating therapy may include administration of one or more of: E2609; MK-8931; LY2886721; AZD3293; semagacestat (LY-450139); avagacestat (BMS-708163); solanezumab; crenezumab; bapineuzumab; BIIB037; CAD106; 8F5 or 5598 or other antibodies raised against Aβ globulomers, e.g., as described by Barghorn et al, “Globular amyloid β-peptide1-42 oligomer—a homogenous and stable neuropathological protein in Alzheimer's disease” J. Neurochem., 2005, 95, 834-847, the entire teachings of which are incorporated herein by reference; ACC-001; V950; Affitrope AD02; and the like.

In several embodiments, the method may include selectively concentrating the soluble, misfolded Aβ protein in one or more of the sample, the incubation mixture, and the detection mixture. The selectively concentrating the soluble, misfolded Aβ protein may include pre-treating the sample prior to forming the incubation mixture. The selectively concentrating the soluble, misfolded Aβ protein may include pre-treating the incubation mixture prior to incubating the incubation mixture. The selectively concentrating the soluble, misfolded Aβ protein may include contacting one or more Aβ specific antibodies to the soluble, misfolded Aβ protein to form a captured soluble, misfolded Aβ protein. The one or more Aβ specific antibodies may include one or more of: 6E10, 4G8, 82E1, A11, X-40/42, and 16ADV. Such antibodies may be obtained as follows: 6E10 and 4G8 (Covance, Princeton, N.J.); 82E1 (IBL America, Minneapolis, Minn.); A11 (Invitrogen, Carlsbad, Calif.); X-40/42 (MyBioSource, Inc., San Diego, Calif.); and 16ADV (Acumen Pharmaceuticals, Livermore, Calif.). The one or more Aβ specific antibodies may include one or more of: an antibody specific for an amino acid sequence of Aβ and an antibody specific for a conformation of the soluble, misfolded Aβ protein. The one or more Aβ specific antibodies may be coupled to a solid phase. The solid phase may include one or more of a magnetic bead and a multiwell plate.

For example, ELISA plates may be coated with the antibodies used to capture Aβ from the patient sample. The antibody-coated ELISA plates may be incubated with a patient sample, unbound materials may be washed off, and the PMCA reaction may be performed. Antibodies may also be coupled to beads. The beads may be incubated with the patient sample and used to separate Aβ-antibody complexes from the remainder of the patient sample.

In various embodiments, the contacting the sample with the monomeric Aβ protein to form the incubation mixture may include contacting a molar excess of the monomeric Aβ protein to the sample including the captured soluble, misfolded Aβ protein. The molar excess of the monomeric Aβ protein may be greater than a total molar amount of Aβ protein monomer included in the captured soluble, misfolded Aβ protein. The incubating the incubation mixture may be effective to cause oligomerization of at least a portion of the monomeric Aβ protein in the presence of the captured soluble, misfolded Aβ protein to form the amplified portion of the soluble, misfolded Aβ protein.

In some embodiments, the protein aggregate may include one or more of: the monomeric Aβ protein, the soluble, misfolded Aβ protein, a captured form of the soluble, misfolded Aβ protein, larger Aβ aggregates, and the like.

In several embodiments, the physically disrupting the incubation mixture may include one or more of: sonication, stirring, shaking, freezing/thawing, laser irradiation, autoclave incubation, high pressure, and homogenization. For example, shaking may include cyclic agitation. The cyclic agitation may be conducted between about 50 rotations per minute (RPM) and 10,000 RPM. The cyclic agitation may be conducted between about 200 RPM and about 2000 RPM. The cyclic agitation may be conducted at about 500 RPM.

In various embodiments, the physically disrupting the incubation mixture may be conducted in each incubation cycle for between about 5 seconds and about 10 minutes, between about 30 sec and about 1 minute, between about 45 sec and about 1 minute, for about 1 minute, and the like. For example, the physically disrupting the incubation mixture may be conducted in each incubation cycle by shaking for one or more of: between about 5 seconds and about 10 minutes, between about 30 sec and about 1 minute, between about 45 sec and about 1 minute, for about 1 minute, and the like. The incubating the incubation mixture may be independently conducted, in each incubation cycle, for a time between about 1 minutes and about 5 hours, between about 10 minutes and about 2 hours, between about 15 minutes and about 1 hour, between about 25 minutes and about 45 minutes, and the like. Each incubation cycle may include independently incubating and physically disrupting the incubation mixture for one or more of: incubating between about 1 minutes and about 5 hours and physically disrupting between about 5 seconds and about 10 minutes; incubating between about 10 minutes and about 2 hours and physically disrupting between about 30 sec and about 1 minute; incubating between about 15 minutes and about 1 hour and physically disrupting between about 45 sec and about 1 minute; incubating between about 25 minutes and about 45 minutes and physically disrupting between about 45 sec and about 1 minute; and incubating about 1 minute and physically disrupting about 1 minute.

The conducting the incubation cycle may be repeated between about 2 times and about 1000 times, between about 5 times and about 500 times, between about 50 times and about 500 times, between about 150 times and about 250 times, and the like. The incubating the incubation mixture being independently conducted, in each incubation cycle, at a temperature in ° C. of about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range between any two of the preceding values, for example, between about 15° C. and about 50° C.

In several embodiments, contacting the sample with the monomeric Aβ protein to form the incubation mixture may be conducted under physiological conditions. Contacting the sample with the monomeric Aβ protein to form the incubation mixture may include contacting the sample with a molar excess of the monomeric Aβ protein. The molar excess may be greater than a total molar amount of Aβ protein monomer included in the soluble, misfolded Aβ protein in the sample. The monomeric Aβ protein and/or the soluble, misfolded Aβ protein may include one or more peptides formed via β- or γ-secretase cleavage of amyloid precursor protein. The monomeric Aβ protein and/or the soluble, misfolded Aβ protein may include one or more of: Abeta40 and Abeta42.

In various embodiments of the methods described herein, the soluble, misfolded Aβ protein may substantially be the soluble, misfolded Aβ aggregate. The amplified portion of misfolded Aβ protein may substantially be one or more of: the amplified portion of the soluble, misfolded Aβ aggregate and the insoluble, misfolded Aβ aggregate. The monomeric, folded Aβ protein may be produced by one of: chemical synthesis, recombinant production, or extraction from non-recombinant biological samples.

In various embodiments, kits for determining a presence of a soluble, misfolded Aβ protein in a sample are provided. The kits may include one or more of a known amount of a monomeric Aβ protein and a known amount of an indicator of the soluble, misfolded Aβ protein. The kits may include instructions. The instructions may direct a user to contact the sample with one or more of the known amount of the monomeric, folded Aβ protein and the known amount of the indicator of the soluble, misfolded Aβ protein to form an incubation mixture. The instructions may direct a user to conduct an incubation cycle two or more times effective to form an amplified portion of misfolded Aβ protein from the monomeric, folded Aβ protein. Each incubation cycle may include incubating the incubation mixture effective to cause misfolding and/or aggregation of at least a portion of the monomeric, folded Aβ protein in the presence of the soluble, misfolded Aβ protein. Each incubation cycle may include physically disrupting the incubation mixture effective to at least partly de-aggregate at least a portion of a misfolded Aβ aggregate present. The instructions may direct a user to determine the presence of the soluble, misfolded Aβ protein in the sample by detecting at least a portion of the amplified portion of misfolded Aβ protein. The soluble, misfolded Aβ protein may include one or more of: a soluble, misfolded Aβ monomer and a soluble, misfolded Aβ aggregate. The amplified portion of misfolded Aβ protein may include one or more of: an amplified portion of the soluble, misfolded Aβ monomer, an amplified portion of the soluble, misfolded Aβ aggregate, and an insoluble, misfolded Aβ aggregate.

In various embodiments, the kits may include the known amount of the monomeric Aβ protein and the known amount of the indicator of the soluble, misfolded Aβ protein. The kits may include one or more of: a multiwall microtitre plate; a microfluidic plate; a shaking apparatus; an incubating apparatus; and a fluorescence measurement apparatus; included either as one or more of the individual plates and apparatuses, as a combination device, and the like. For example, a shaking microplate reader may be used to perform cycles of incubation and shaking and automatically measure the ThT fluorescence emission during the course of an experiment (e.g., FLUOstar OPTIMA, BMG LABTECH Inc., Cary, N.C.).

In several embodiments of the kits, an indicating state and a non-indicating state of the indicator of misfolded Aβ protein may be characterized by a difference in fluorescence, light absorption or radioactivity depending on the specific indicator. The instructions may direct the user to determine the presence of the soluble, misfolded Aβ protein in the sample by fluorescence, light absorption or radioactivity, or other forms of spectroscopy, depending on the specific indicator.

In some embodiments of the kits, the indicator of misfolded Aβ protein may include one or more of: Thioflavin T, Congo Red, m-I-Stilbene, Chrysamine G, PIB, BF-227, X-34, TZDM, FDDNP, MeO-X-04, IMPY, NIAD-4, luminescent conjugated polythiophenes, a fusion with a fluorescent protein such as green fluorescent protein and yellow fluorescent protein, derivatives thereof, and the like. The monomeric, folded Aβ protein may include one or more of a covalently incorporated radioactive amino acid, a covalently incorporated, isotopically labeled amino acid, and a covalently incorporated fluorophore.

In various embodiments of the kits, the instructions may direct a user to conduct any of the methods described herein. For example, the instructions may include directions to the user to determine an amount of the soluble, misfolded Aβ protein in the sample. The instructions may direct the user to detect the soluble, misfolded Aβ protein in the detection mixture by conducting one or more of: a Western Blot assay, a dot blot assay, an enzyme-linked immunosorbent assay (ELISA), a thioflavin T binding assay, a Congo Red binding assay, a sedimentation assay, electron microscopy, atomic force microscopy, surface plasmon resonance, spectroscopy, and the like.

The instructions may direct the user to detect the soluble, misfolded Aβ protein in the detection mixture by contacting the detection mixture with a protease; and detecting the soluble, misfolded Aβ protein in the detection mixture using anti-misfolded protein antibodies in one or more of: a Western Blot assay, a dot blot assay, and an ELISA.

In several embodiments of the kits, the instructions may direct the user to take the sample from a subject. The instructions may include directing the user to determine the presence of AD in the subject according to the presence of the soluble, misfolded Aβ protein in the sample. The presence of the soluble, misfolded Aβ protein in the sample may be determined compared to a control sample taken from a control subject. The instructions may direct the user to determine the presence of AD in the subject by comparing the amount of the soluble, misfolded Aβ protein in the sample to a predetermined threshold amount. The instructions may direct the user to obtain the sample including one or more of: amniotic fluid; bile; blood; cerebrospinal fluid; cerumen; skin; exudate; feces; gastric fluid; lymph; milk; mucus, e.g. nasal secretions; mucosal membrane, e.g., nasal mucosal membrane; peritoneal fluid; plasma; pleural fluid; pus; saliva; sebum; semen; sweat; synovial fluid; tears; and urine. The instructions may direct the user to determine a progression or homeostasis of AD in the subject by comparing the amount of the soluble, misfolded Aβ protein in the sample to an amount of the soluble, misfolded Aβ protein in a comparison sample taken from the subject at a different time compared to the sample.

The instructions may direct the user to the user to selectively concentrate the soluble, misfolded Aβ protein in one or more of the sample, the incubation mixture and the detection mixture. For example, the kit may include one or more Aβ specific antibodies configured to selectively concentrate or capture the soluble, misfolded Aβ protein. The one or more Aβ specific antibodies may include one or more of: an antibody specific for an amino acid sequence of Aβ or an antibody specific for a conformation of the soluble, misfolded Aβ protein. The one or more Aβ specific antibodies may include one or more of: 6E10, 4G8, 82E1, A11, X-40/42, and 16ADV. The instructions may direct the user to selectively concentrate the soluble, misfolded Aβ protein by contacting the one or more Aβ specific antibodies to the soluble, misfolded Aβ protein to form a captured soluble, misfolded Aβ protein. The one or more Aβ specific antibodies may be provided coupled to a solid phase. The solid phase may include one or more of a magnetic bead and a multiwell plate.

In various embodiments of the kit, the instructions for physically disrupting the incubation mixture may direct the user to employ one or more of: sonication, stirring, shaking, freezing/thawing, laser irradiation, autoclave incubation, high pressure, homogenization, and the like. The instructions may direct the user to conduct cyclic agitation according to any RPM range described herein, for example, between about 50 RPM and 10,000 RPM. The instructions may direct the user to conduct the physical disruption in each incubation cycle according to any time range described herein, for example, between about 5 seconds and about 10 minutes. The instructions may direct the user to incubate the incubation mixture in each incubation cycle according to any time range described herein, for example, for a time between about 1 minutes and about 5 hours. The instructions for conducting the incubation cycle may direct the user to conduct the incubation cycle for any number of repetitions described herein, for example, between about 2 times and about 1000 times. Instructions for conducting the incubation cycle may include directions to a user to incubate at a temperature between about 15° C. and about 50° C.

In various embodiments of the kits described herein, the soluble, misfolded Aβ protein may substantially be the soluble, misfolded Aβ aggregate. The amplified portion of misfolded Aβ protein may substantially be one or more of: the amplified portion of the soluble, misfolded Aβ aggregate and the insoluble, misfolded Aβ aggregate. The monomeric, folded Aβ protein may be produced by one of: chemical synthesis, recombinant production, or extraction from non-recombinant biological samples.

### EXAMPLES

### Example 1

### Preparation of Synthetic Aβ Oligomers

Aβ1-42 was synthesized using solid-phase N-tert-butyloxycarbonyl chemistry at the W. Keck Facility at Yale University and purified by reverse-phase HPLC. The final product was lyophilized and characterized by amino acid analysis and mass spectrometry. To prepare stock solutions free of aggregated, misfolded Aβ protein, aggregates were dissolved high pH and filtration through 30 kDa cut-off filters to remove remaining aggregates. To prepare different types of aggregates, solutions of seed-free Aβ1-42 (10 μM) were incubated for different times at 25° C. in 0.1 M Tris-HCl, pH 7.4 with agitation. This preparation contained a mixture of Aβ monomers as well as fibrils, protofibrils and soluble, misfolded Aβ protein in distinct proportions depending on the incubation time. The degree of aggregation was characterized by ThT fluorescence emission, electron microscopy after negative staining, dot blot studies with the A11 conformational antibody and western blot after gel electrophoresis using the 4G8 anti-Aβ antibody.

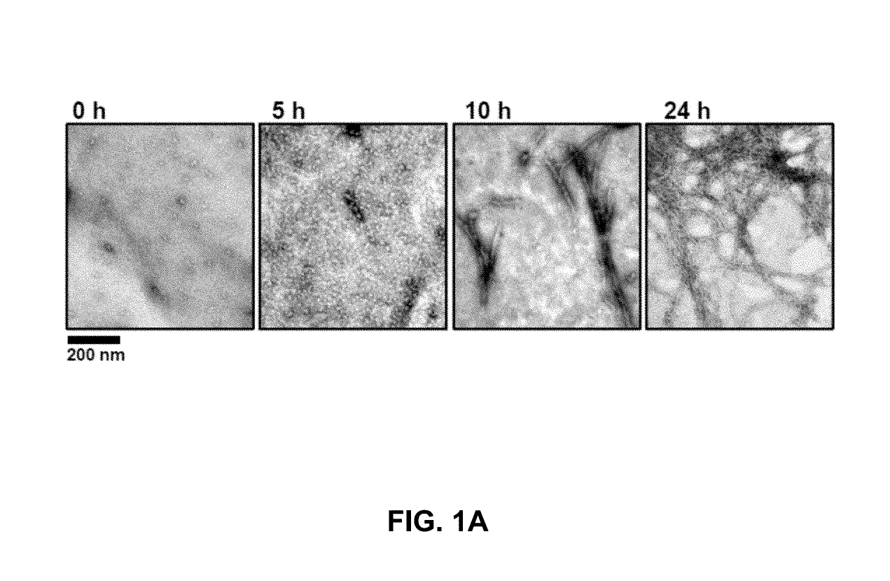

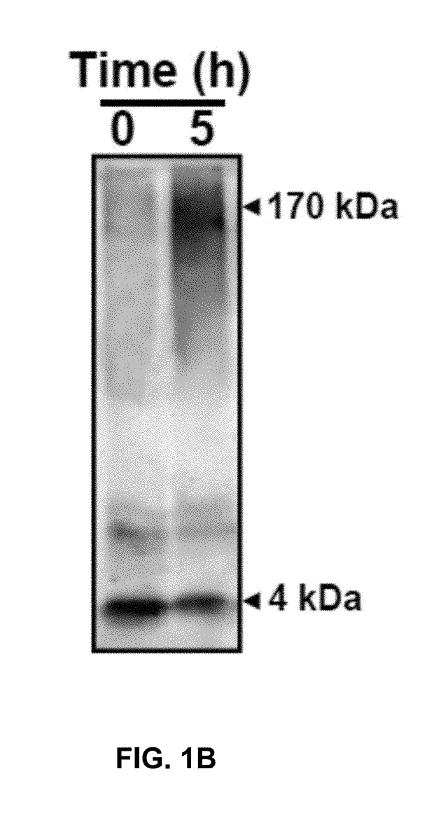

A mixture of Aβ oligomers of different sizes were generated during the process of fibril formation. Specifically, soluble, misfolded Aβ protein was prepared by incubation of monomeric synthetic Aβ1-42 (10 μM) at 25° C. with stirring. After 5 h of incubation, an abundance of soluble, misfolded Aβ protein, globular in appearance, was observed by electron microscopy after negative staining, with only a small amount of protofibrils and fibrils observed. At 10 h there are mostly protofibrils and at 24 h, a large amount of long fibrils are observed. FIG. 1A shows electron micrographs taken at 0 h, 5 h, 10 h, and 24 h of incubation.

The soluble, misfolded Aβ protein aggregates tested positive using A11 anti-oligomer specific antibody according to the method of Kayed, et al. “Common structure of soluble amyloid oligomers implies common mechanism of pathogenesis,” Science 2003, 300, 486-489. After further incubation at 10 h and 24 h, protofibrillar and fibrillar structures were observed. The size of the aggregates was determined by filtration through filters of defined pore size and western blotting after SDS-PAGE separation. Soluble, misfolded Aβ protein formed by incubation for 5 h was retained in filters of 30 kDa cut-off and passed through 1000 kDa cutoff filters. FIG. 1B is a western blot of soluble, misfolded Aβ protein aggregates. Electrophoretic separation of this soluble, misfolded Aβ protein showed that the majority of the material migrated as ˜170 kDa SDS-resistant aggregates, with a minor band at 17 kDa.

### Example 2

### Aβ-PMCA Detects Synthetic Aβ Oligomers

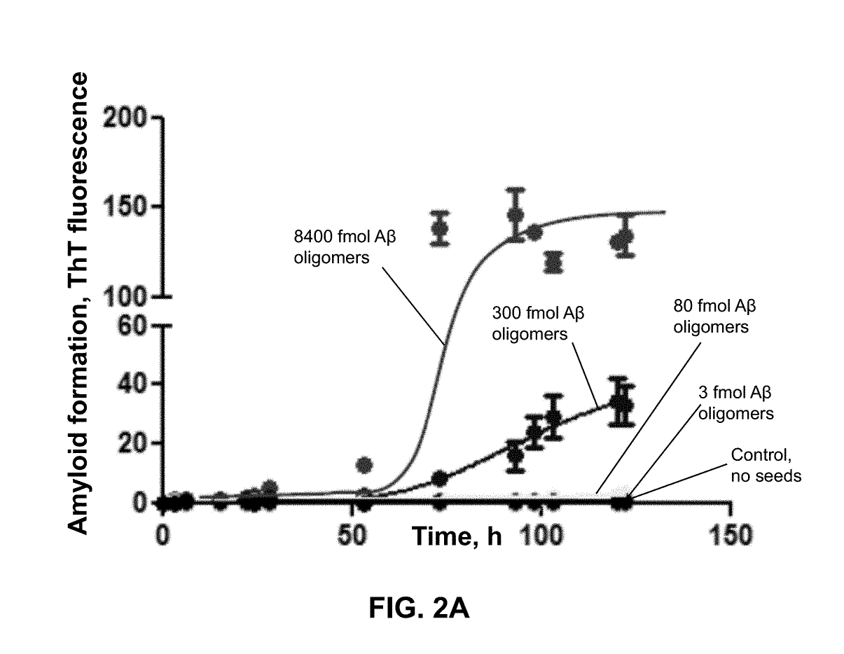

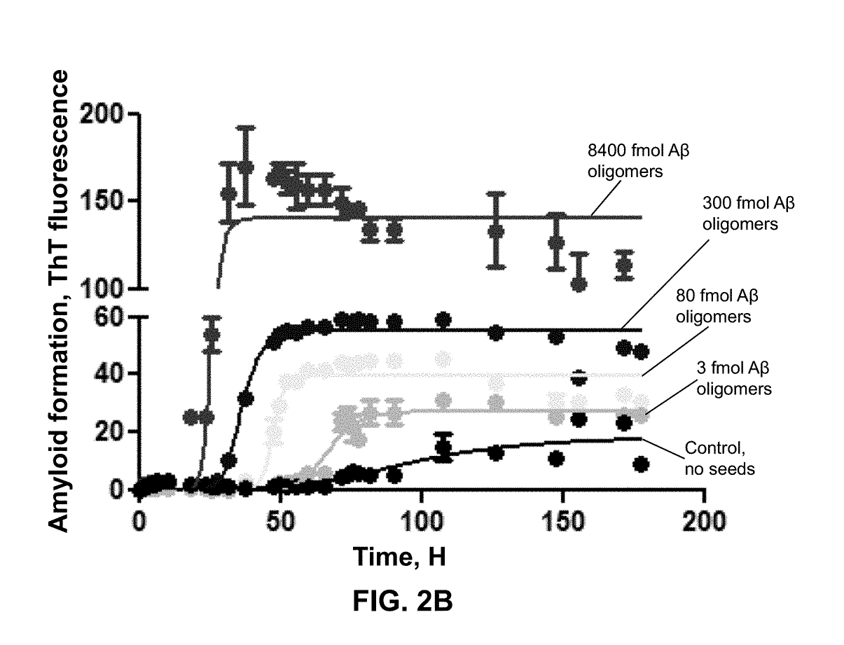

EXAMPLE 2A. Seeding of Aβ aggregation was studied by incubating a solution of seed-free Aβ1-42 in the presence of Thioflavin T with or without different quantities of synthetic soluble, misfolded Aβ protein (Control (no Aβ oligomer); or 3, 80, 300, and 8400 femtomolar in synthetic soluble, misfolded Aβ protein). Aβ-PMCA general procedure: Solutions of 2 μM aggregate-free Aβ1-42 in 0.1 M Tris-HCl pH 7.4 (200 μL total volume) were placed in opaque 96-wells plates and incubated alone or in the presence of synthetic Aβ aggregates (prepared by incubation over 5 h as described in EXAMPLE 1) or 40 μL of CSF aliquots. Samples were incubated in the presence of 5 μM Thioflavin T (ThT) and subjected to cyclic agitation (1 min at 500 rpm followed by 29 min without shaking) using an Eppendorf thermomixer, at a constant temperature of 22° C. At various time points, ThT fluorescence was measured in the plates at 485 nm after excitation at 435 nm using a plate spectrofluorometer. FIG. 2A is a graph of amyloid formation (without cyclic amplification) versus time as measured by Thioflavin T fluorescence, using the indicated femtomolar concentration of synthetic soluble, misfolded Aβ protein seeds. The peptide concentration, temperature and pH of the buffer were monitored to control the extent of the lag phase and reproducibility among experiments. Under these conditions, no spontaneous Aβ aggregation was detected during the time in which the experiment was performed (125 h). Aggregation of monomeric Aβ1-42 protein was observed in the presence of 0.3 to 8.4 fmol of the synthetic soluble, misfolded Aβ protein of EXAMPLE 1.

EXAMPLE 2B: Amplification cycles, combining phases of incubation and physical disruption were employed. The same samples as in FIG. 2A were incubated with cyclic agitation (1 min stirring at 500 rpm followed by 29 min without shaking). Aggregation was measured over time by the thioflavin T (ThT) binding to amyloid fibrils using a plate spectrofluorometer (excitation: 435; emission: 485 nm). Graphs show the mean and standard error of 3 replicates. The concentration of Aβ oligomers was estimated assuming an average molecular weight of 170 kDa. FIG. 2B is a graph showing amplification cycle-accelerated amyloid formation measured by ThT fluorescence as a function of time for various concentrations of the synthetic soluble, misfolded Aβ protein of EXAMPLE 1. Under these conditions, the aggregation of monomeric Aβ1-42 protein induced by 8400, 300, 80 and 3 fmol of the synthetic soluble, misfolded Aβ protein was clearly faster and more easily distinguished from that observed in the absence of the synthetic soluble, misfolded Aβ protein. This result indicates the detection limit, under these conditions, is 3 fmol of soluble, misfolded Aβ protein or less in a given sample.

### Example 3

### Aβ-PMCA Detects Misfolded Aβ in the Cerebrospinal Fluid of AD Patients

Aliquots of CSF were obtained from 50 AD patients, 39 cognitively normal individuals affected by non-degenerative neurological diseases (NND), and 37 patients affected by non-AD neurodegenerative diseases including other forms of dementia (NAND). Test CSF samples were obtained from 50 patients with the diagnosis of probable AD as defined by the DSM-IV and the NINCDS-ADRA guidelines (McKhann et al., 1984) and determined using a variety of tests, including routine medical examination, neurological evaluation, neuropsychological assessment, magnetic resonance imaging and measurements of CSF levels of Aβ1-42, total Tau and phospho-Tau. The mean age of AD patients at the time of sample collection was 71.0±8.1 years (range 49-84). Control CSF samples were obtained from 39 patients affected by non-degenerative neurological diseases (NND), including 12 cases of normal pressure hydrocephalus, 7 patients with peripheral neuropathy, 7 with diverse forms of brain tumor, 2 with ICTUS, 1 with severe cephalgia, 3 with encephalitis, 1 with hypertension and 6 with unclear diagnosis. The mean age at which CSF samples were taken from this group of patients was 64.6±14.7 years (range 31-83). Control CSF samples were also taken from 37 individuals affected by non-AD neurodegenerative diseases (NAND), including 10 cases of fronto-temporal dementia (5 behavioral and 5 language variants), 6 patients with Parkinson's disease (including 4 associated with dementia and 1 with motor neuron disease), 6 with progressive supranuclear palsy, 6 with spinocerebellar ataxia (1 associated with dementia), 4 with amyotrophic lateral sclerosis, 2 with Huntington's disease, 1 with MELAS, 1 with Lewy body dementia, and 1 with vascular dementia. The mean age at sample collection for this group was 63.8±11.1 years (range 41-80). CSF samples were collected in polypropylene tubes following lumbar puncture at the L4/L5 or L3/L4 interspace with atraumatic needles after one night fasting. The samples were centrifuged at 3,000 g for 3 min at 4° C., aliquoted and stored at −80° C. until analysis. CSF cell counts, glucose and protein concentration were determined. Albumin was measured by rate nephelometry. To evaluate the integrity of the blood brain barrier and the intrathecal IgG production, the albumin quotient (CSF albumin/serum albumin) X 10³ and the IgG index (CSF albumin/serum albumin)/(CSF IgG/serum IgG) were calculated. The study was conducted according to the provisions of the Helsinki Declaration and was approved by the Ethics Committee.

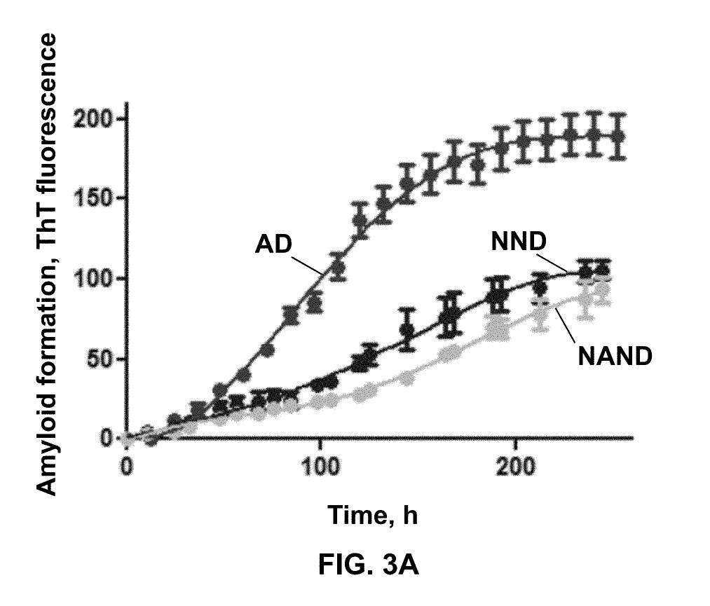

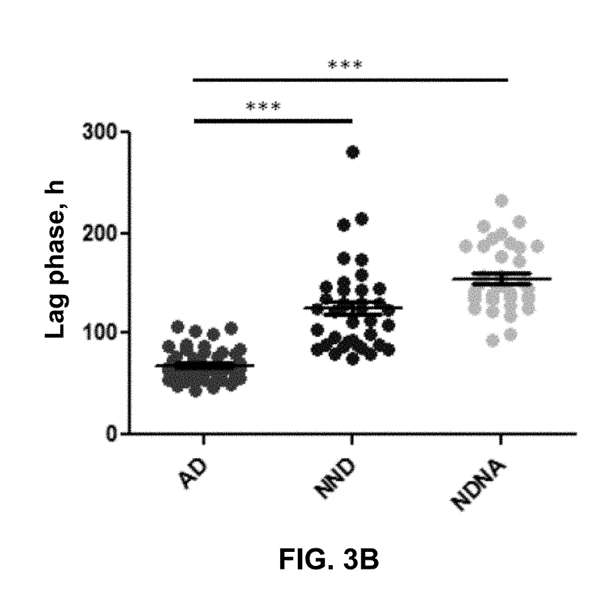

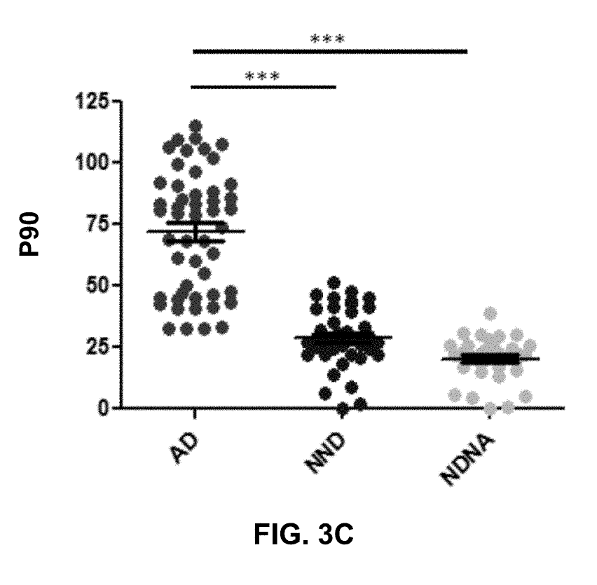

The experiments as well as the initial part of the analysis were conducted blind. FIG. 3A is a graph of amyloid formation versus time, measured as a function of ThT fluorescence labeling, showing the average kinetics of Aβ aggregation of 5 representative samples from the AD, NND, and NAND groups.

The results indicate that CSF from AD patients significantly accelerates Aβ aggregation as compared to control CSF (P<0.001). The significance of the differences in Aβ aggregation kinetics in the presence of human CSF samples was analyzed by one-way ANOVA, followed by the Tukey's multiple comparison post-test. The level of significance was set at P<0.05. The differences between AD and samples from the other two groups were highly significant with P<0.001 (***).

FIG. 3B is a graph of the lag phase time in h for samples from the AD, NND, and NAND groups. To determine the effect of individual samples on Aβ aggregation, the lag phase was estimated, defined as the time to ThT fluorescence larger than 40 arbitrary units after subtraction of a control buffer sample. This value was selected considering that it corresponds to ˜5 times the reading of the control buffer sample.

FIG. 3C is a graph showing the extent of amyloid formation obtained after 180 Aβ-PMCA cycles, i.e. 90 h of incubation (P90). Comparison of the lag phase and P90 among the experimental groups reveals a significant difference between AD and control samples from individuals with non-degenerative neurological diseases or with non-AD neurodegenerative diseases. Further, no correlation was detected between the aggregation parameters and the age of the AD patients, which indicates that the levels of the marker corresponds to aggregated Aβ protein in patient CSF, and not patient age.

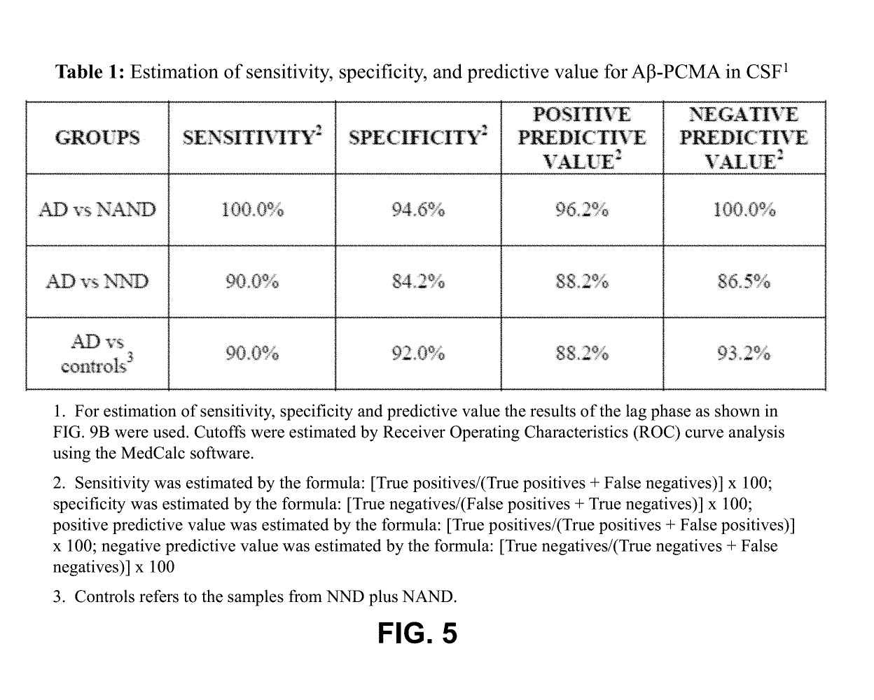

FIG. 5, Table 1 shows estimations of the sensitivity, specificity and predictive value of the Aβ-PMCA test, calculated using the lag phase numbers.

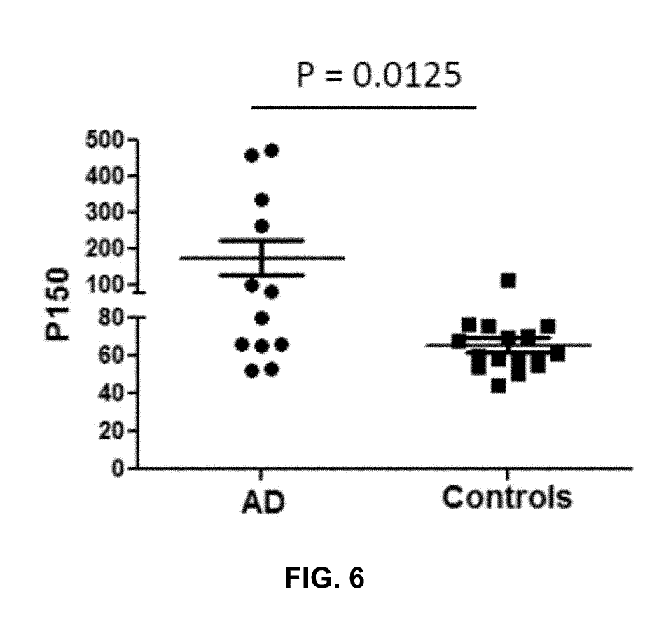

To study reproducibility, an experiment similar to the one shown in FIGS. 3A-C was independently done with different samples, reagents and a new batch of Aβ peptide as substrate for Aβ-PMCA. The extent of amyloid formation obtained after 300 Aβ-PMCA cycles, i.e. 150 h of incubation (P150), was measured in each patient. The control group includes both people affected by other neurodegenerative diseases and non-neurologically sick patients. Data for each sample represent the average of duplicate tubes. Statistical differences were analyzed by student-t test. FIG. 6 is a graph of the lag phase time in h for samples obtained after 300 Aβ-PMCA cycles, i.e. 150 h of incubation (P90).

During the course of the study an entire set of CSF samples coming from a fourth location did not aggregate at all, even after spiking with large concentrations of synthetic oligomers. It is expected that reagent contamination during sample collection interfered with the assay.

The differences in aggregation kinetics between different samples were evaluated by the estimation of various different kinetic parameters, including the lag phase, A50, and P90. Lag phase is defined as the time required to reach a ThT fluorescence higher than 5 times the background value of the buffer alone. The A50 corresponds to the time to reach 50% of maximum aggregation. P90 corresponds to the extent of aggregation (measured as ThT fluorescence) at 90 h. Sensitivity, specificity and predictive value were determined using this data, with cutoff thresholds determined by Receiver Operating Characteristics (ROC) curve analysis, using MedCalc software (MedCalc Software, Ostend, Belgium).

### Example 4

### Determination of Threshold Values of Misfolded Aβ for Aβ-PMCA Detection of Aβ in CSF

In support of FIG. 5, TABLE 1, sensitivity, specificity and predictive value were determined using the lag phase data, with cutoff thresholds determined by Receiver Operating Characteristics (ROC) curve analysis, using the MedCalc software (version 12.2.1.0, MedCalc, Belgium). As shown in FIG. 5, TABLE 1, a 90.0% sensitivity and 84.2% specificity was estimated for the control group consisting of age-matched individuals with non-degenerative neurological diseases. By contrast, for the clinically more relevant differentiation of AD from other neurodegenerative diseases including other forms of dementia, 100% sensitivity and 94.6% specificity was estimated. This ability of Aβ-PMCA to distinguish AD from other forms of neurodegenerative diseases is clinically significant. The overall sensitivity and specificity considering all control individuals was 90% and 92%, respectively.

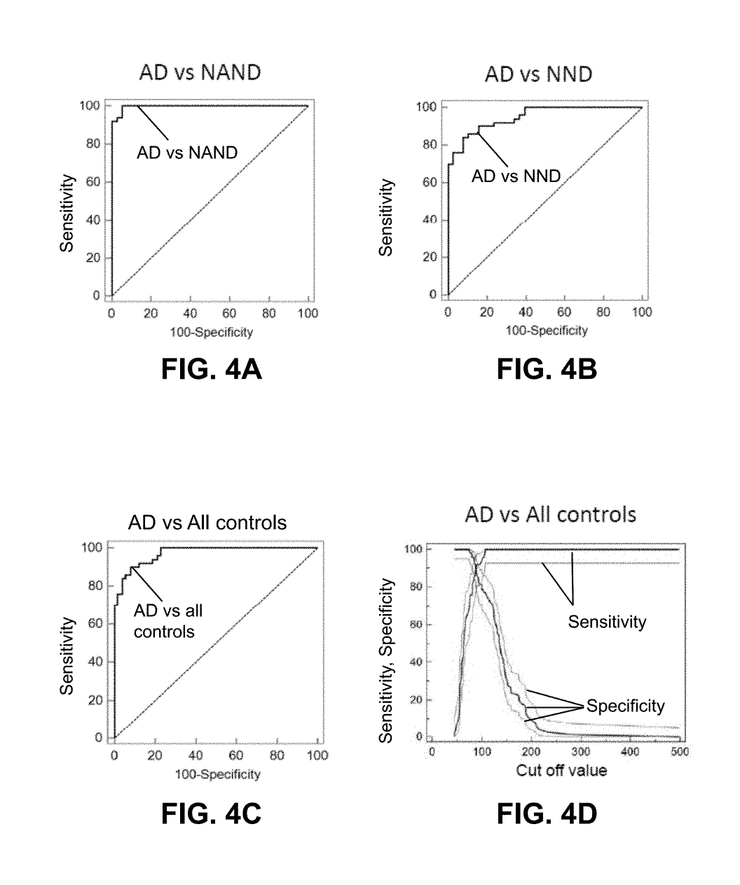

To evaluate the performance of the Aβ-PMCA test to distinguish AD patients from controls, the true positive rate (sensitivity) was plotted as a function of the false positive rate (specificity) for different cut-off points. For this analysis the lag phase values for AD vs NAND (FIG. 4A), AD vs NND (FIG. 4B) and AD vs All control samples (FIG. 4C) was used. The performance of the test, estimated as the area under the curve was 0.996±0.0033, 0.95±0.020 and 0.97±0.011 for the comparison of AD with NAND, NND and all controls, respectively. Statistical analysis was done using the MedCalc ROC curve analysis software (version 12.2.1.0) and the result indicated that the test can distinguish AD from the controls with a P<0.0001. To estimate the most reliable cut-off point for the different set of group comparisons, sensitivity (blue line) and specificity (red line) were plotted for each cut-off value (FIG. 4D). The graph shows the curve and the 95% confidence intervals for the AD vs all control samples (including NAND and NND groups). These cut-off values were used to estimate sensitivity, specificity and predictive value in FIG. 5, Table 1.

### Example 5

### Aβ-Oligomer Immunodepletion Removes Aβ Seeds in Human Cerebrospinal Fluid and Confirms Aβ-PMCA Detects Soluble Misfolded Aβ Protein in AD CSF

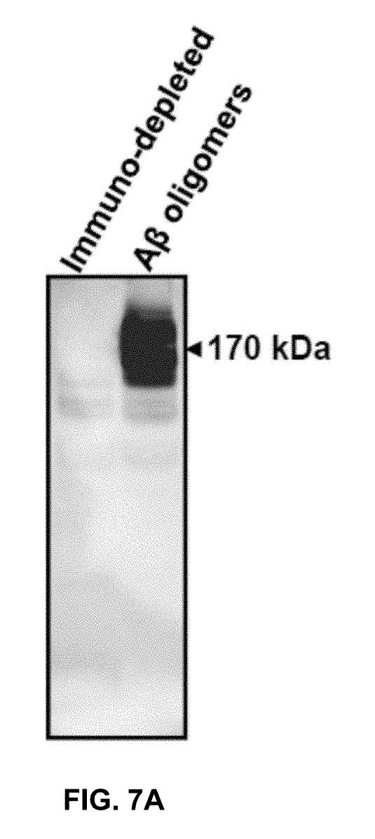

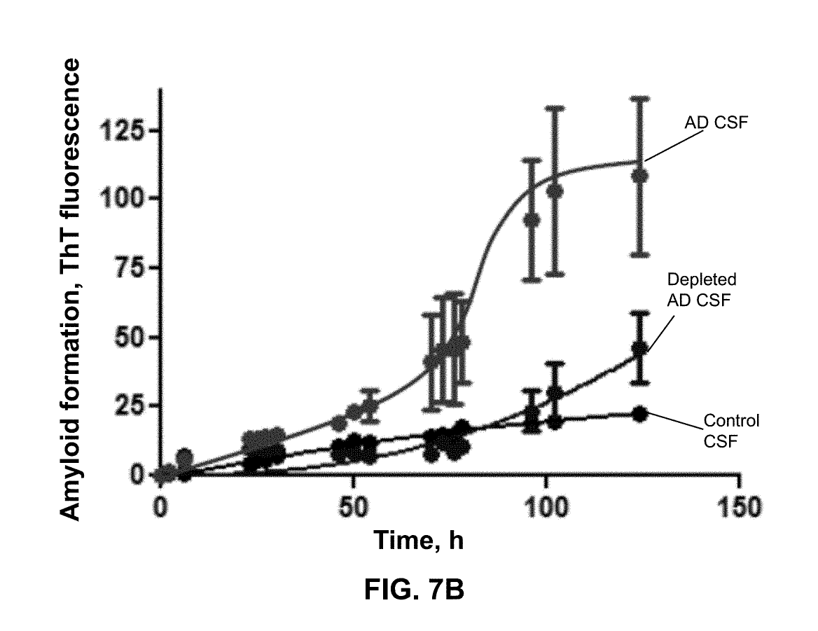

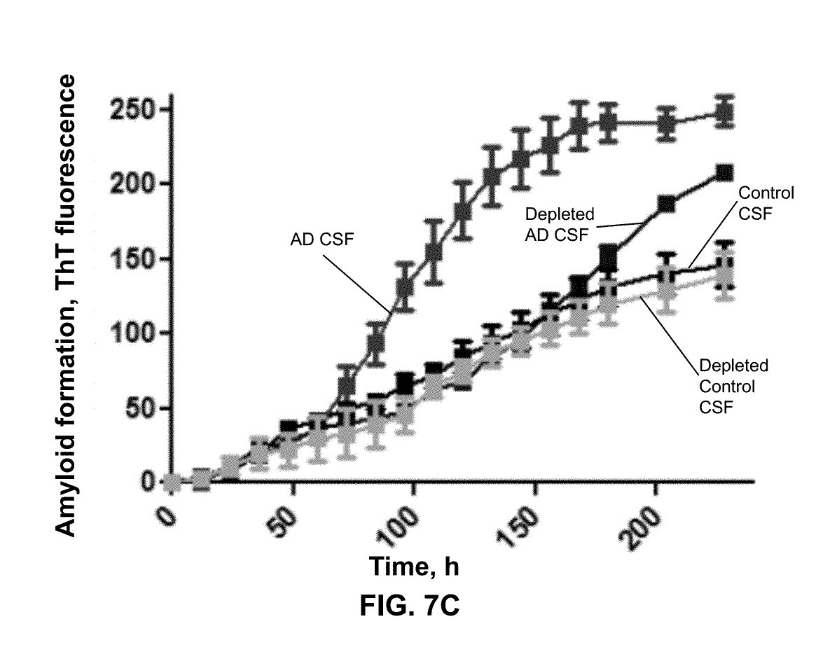

Immunodepletion experiments were performed to confirm that Aβ-PMCA detects a seeding activity associated to soluble, misfolded Aβ protein present in CSF. The methodology for efficient immunodepletion of soluble, misfolded Aβ protein was first optimized by using synthetically prepared soluble, misfolded Aβ protein. Immunodepletion was performed by incubation with dynabeads conjugated with a mixture of antibodies recognizing specifically the sequence of Aβ (4G8) and conformational (A11) antibodies. FIG. 7A is a western blot showing results of immunodepletion using synthetically prepared Aβ oligomers spiked into human CSF. Soluble, misfolded Aβ protein was efficiently removed by this immunodepletion.

FIGS. 7A and 7B are graphs of amyloid formation versus time as measured by Thioflavin T fluorescence, demonstrating that seeding activity in AD CSF is removed by soluble, misfolded Aβ protein immuno-depletion. Samples of AD CSF before or after immunodepletion with 4G8 and A11 antibodies were used to seed Aβ aggregation in the Aβ-PMCA assay. Immunodepletion was applied to 3 AD CSF. FIG. 7B is a graph showing the kinetics of control and immunodepleted CSF samples. FIG. 7B shows that for immunodepleted AD CSF, the kinetics of Aβ aggregation in the Aβ-PMCA reaction was comparable to that observed in control CSF samples, and both were significantly different from the aggregation observed with AD CSF prior to immunodepletion. FIG. 7C is a graph showing the kinetics of control and immunodepleted CSF samples, depleted only with the A11 conformational antibody and aggregation monitored by Aβ-PMCA assay. FIG. 7C shows similar results, obtained using AD CSF immunodepleted using the A11 conformational antibody, which specifically recognizes, misfolded Aβ. These results confirm that Aβ-PMCA detects soluble, misfolded β protein in AD CSF.

### Example 6

### Solid Phase Immuno Capturing

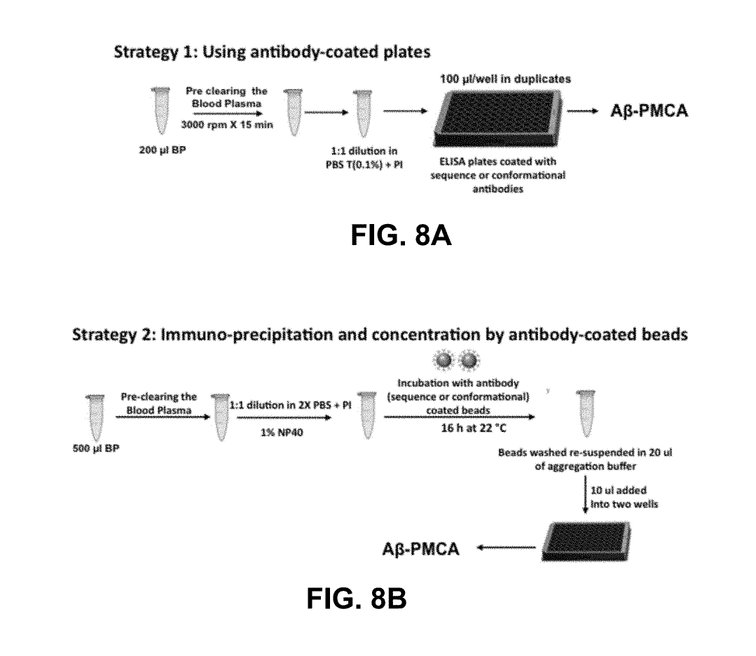

FIGS. 8A and 8B are schematic representations of two solid phase methods used to capture soluble, misfolded Aβ protein from complex samples such as blood plasma. Strategy 1 employed ELISA plates pre-coated with specific antibodies bound to a solid phase on the ELISA plate. After washing the plates, the Aβ-PMCA reaction was carried out in the same plates. Strategy 2 used magnetic beads as the solid phase coated with specific antibodies. This approach provided concentration of the samples.

### Example 7

### Specificity of Immuno Capturing

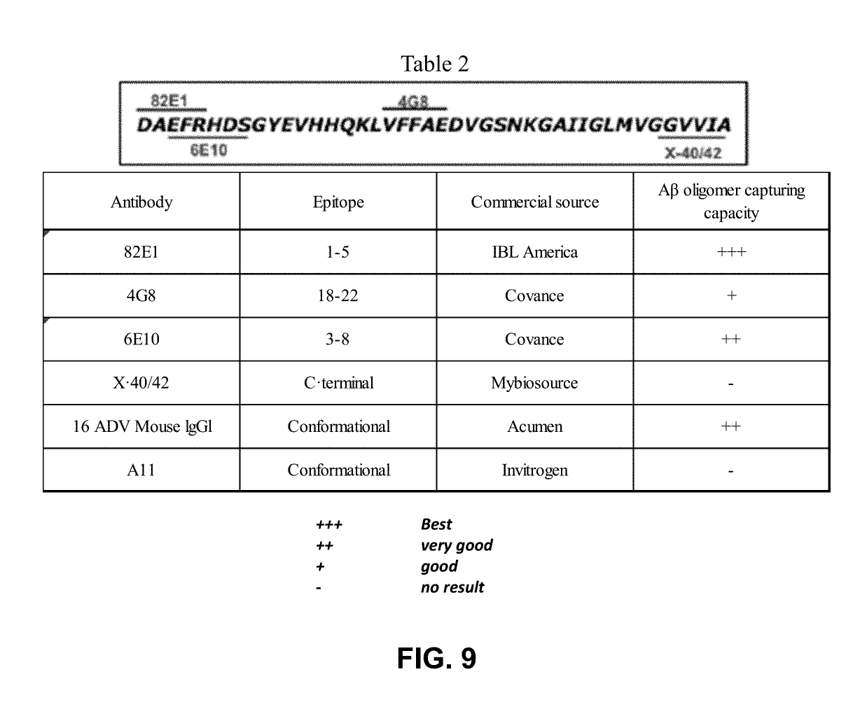

FIG. 9 shows Table 2, demonstrating the ability of specific antibodies to capture the Aβ oligomers. The top panel shows a schematic representation of the epitope recognition site on the Aβ protein of the diverse sequence antibodies used in this study. Table 2 in FIG. 9 demonstrates the efficiency of different sequence or conformational antibodies to capture Aβ oligomers. The capacity to capture oligomers was measured by spiking synthetic Aβ oligomers in healthy human blood plasma and detection by Aβ-PMCA. The symbols indicate that the detection limits using the different antibodies were: <12 fmol (+++); between 10-100 fmol (++); >1 pmol (+) and not significantly higher than without capturing reagent (−).

### Example 8

### Detection of Aβ Oligomers Spiked in Human Plasma

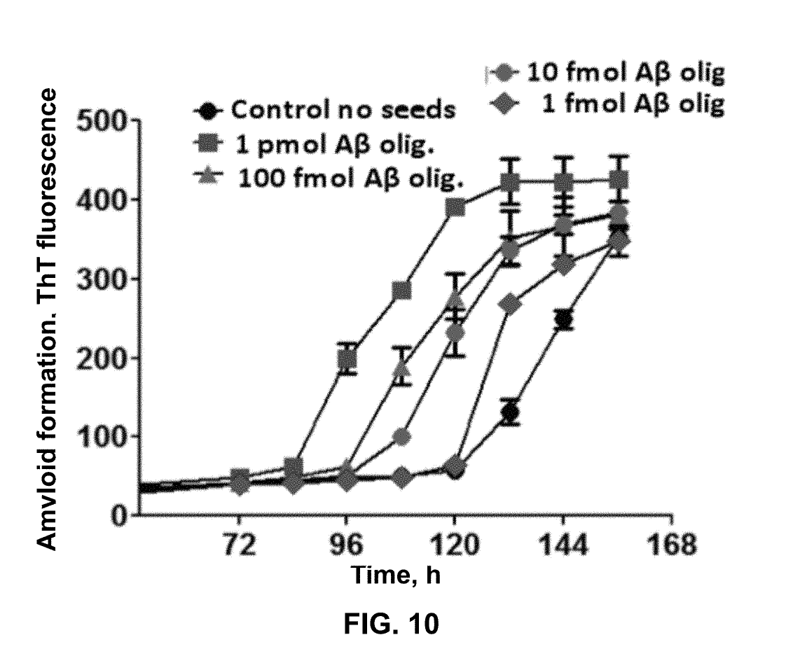

FIG. 10 is a graph of amyloid formation versus time as measured by Thioflavin T fluorescence showing detection of soluble, misfolded Aβ protein spiked in human plasma. ELISA plates pre-coated with protein G were coated with an anti-conformational antibody (16ADV from Acumen). Thereafter, plates were incubated with human blood plasma (100 μl) as such (control) or spiked with different concentrations of synthetic soluble, misfolded Aβ protein. After incubation, plates were subjected to Aβ-PMCA (29 min incubation and 30 s shaking) in the presence of Aβ40 monomer (2 μM) and ThT (5 μM). Amyloid formation was measured by Thioflavin fluorescence. FIG. 10 is representative of several experiments done with 3 different antibodies which worked similarly.

### Example 9

### Capturing of Soluble Misfolded Aβ from AD Patient Samples Vs Controls

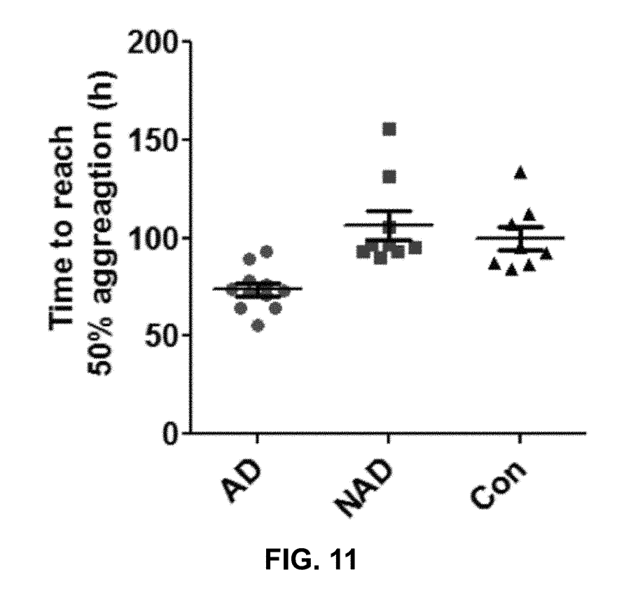

FIG. 11 is a graph showing time to reach 50% aggregation in an Aβ-PMCA assay in plasma samples from AD patients and controls. Blood plasma samples from patients affected by AD, non-AD neurodegenerative diseases (NAD), and healthy controls were incubated with anti-Aβ antibody (82E1) coated beads. Aβ-PMCA was carried out as described in EXAMPLE 2. The time needed to reach 50% aggregation was recorded in individual patients in each group. Differences were analyzed by one-way ANOVA followed by the Tukey's post-hoc test. ROC analysis of this data showed a 82% sensitivity and 100% specificity for correctly identifying AD patients from controls.

### Example 10

### Sonication and Shaking are Effective with Various Detection Methods

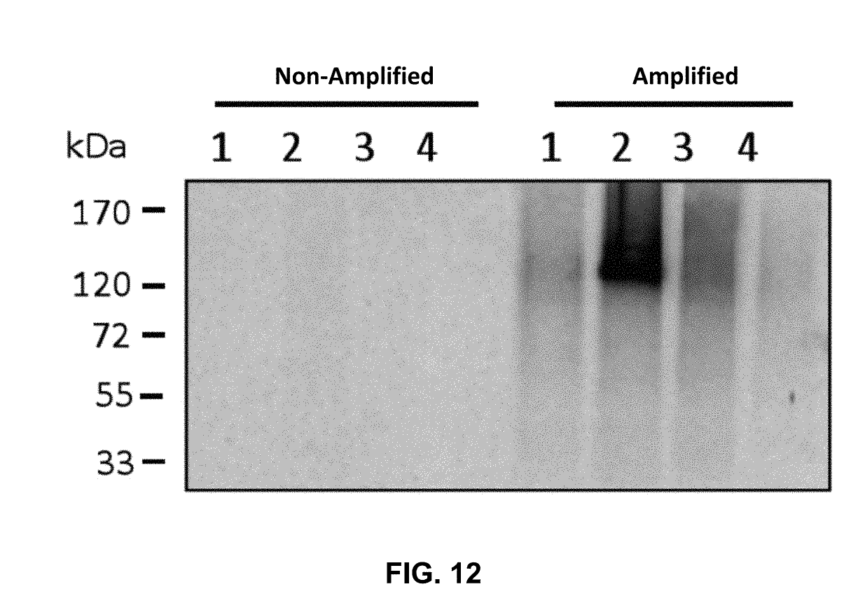

FIG. 12 is a western blot showing the results of amplification of Aβ aggregation by using sonication instead of shaking as a mean to fragment aggregates. The experiment was done in the presence of distinct quantities of synthetic Aβ oligomers. Samples of 10 μg/ml of seed-free monomeric Aβ1-42 were incubated alone (lane 1) or with 300 (lane 2), 30 (lane 3) and 3 (lane 4) fmols of, misfolded Aβ. Samples were either frozen without amplification (non-amplified) or subjected to 96 PMCA cycles (amplified), each including 30 min incubation followed by 20 sec sonication. Aggregated Aβ was detected by western blot using anti-Aβ antibody after treatment of the samples with proteinase K (PK). In our experiments, it was observed that detection using thioflavin T fluorescence was not compatible with sonication, but works very well with shaking as a physical disruption method. FIG. 12 shows that using a different detection method for the Aβ aggregates, in this case Western Blotting, sonication works as well as shaking.

### Example 11

### Production of Monomeric Aβ as PMCA Substrate

Seed-free monomeric Aβ was obtained by size exclusion chromatography. Briefly, an aliquot of a 1 mg/mL peptide solution prepared in dimethylsulfoxide was fractionated using a Superdex 75 column eluted at 0.5 mL/min with 10 mM sodium phosphate at pH 7.5. Peaks will be detected by UV absorbance at 220 nm. The peak corresponding to 4-10 kDa molecular weight containing monomer/dimmers of Aβ was collected and concentration determined by amino acid analysis. Samples were stored lyophilized at −80° C.

### Example 12

### Production and Purification of Aβ

E. coli cells harboring pET28 GroES-Ub-Aβ42 plasmid were grown in Luria broth (LB) at 37° C., and expression was induced with 0.4 mM IPTG. After 4 h, cells were harvested and lysed in a lysis buffer (20 mM Tris-Cl, pH 8.0, 10 mM NaCl, 0.1 mM PMSF, 0.1 mM EDTA and 1 mM β-mercaptoethanol) and centrifuged at 18,000 rpm for 30 min. Inclusion bodies were re-suspended in a resuspension buffer (50 mM Tris-Cl, pH 8.0, 150 mM NaCl, and 1 mM DTT) containing 6 M urea. Insoluble protein was removed by centrifugation at 18,000 rpm for 30 min. The supernatant containing GroES-Ub-Aβ42 fusion protein will be collected. To cleave off Aβ42 from fusion protein, the fusion protein was diluted 2-fold with resuspension buffer and treated with recombinant de-ubiquinating enzyme (Usp2cc) 1:100 enzyme to substrate molar ratio at 37° C. for 2 h. After that, samples was loaded on a C18 column (25 mm×250 mm, Grace Vydac, USA). Aβ42 was purified with a solvent system buffer 1 (30 mM ammonium acetate, pH 10, 2% acetonitrile) and buffer 2 (70% acetonitrile) at a flow rate 10 ml/min using a 20-40% linear gradient of buffer 2 over 35 min. Purified Aβ42 was lyophilized and stored at −80° C., until use.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. To the extent that the term “substantially” is used in the specification or the claims, it is intended to mean that the identified components have the relation or qualities indicated with degree of error as would be acceptable in the subject industry.

As used in the specification and the claims, the singular forms “a,” “an,” and “the” include the plural unless the singular is expressly specified. For example, reference to “a compound” may include a mixture of two or more compounds, as well as a single compound.

As used herein, the term “about” in conjunction with a number is intended to include ±10% of the number. In other words, “about 10” may mean from 9 to 11.

As used herein, the terms “optional” and “optionally” mean that the subsequently described circumstance may or may not occur, so that the description includes instances where the circumstance occurs and instances where it does not.

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 cells refers to groups having 1, 2, or 3 cells. Similarly, a group having 1-5 cells refers to groups having 1, 2, 3, 4, or 5 cells, and so forth. While various aspects and embodiments have been disclosed herein, other aspects and embodiments will be apparent to those skilled in the art.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

The various aspects and embodiments disclosed herein are for purposes of illustration and are not intended to be limiting, with the true scope and spirit being indicated by the following claims.

1. A method for determining a presence of a soluble, misfolded Aβ protein in a sample, comprising:
contacting the sample with a monomeric, folded Aβ protein to form an incubation mixture;
conducting an incubation cycle two or more times on the incubation mixture effective to form an amplified portion of misfolded Aβ protein from the monomeric, folded Aβ protein, each incubation cycle comprising:
incubating the incubation mixture effective to cause misfolding and/or aggregation of at least a portion of the monomeric, folded Aβ protein in the presence of the soluble, misfolded Aβ protein;
physically disrupting the incubation mixture effective to at least partly de-aggregate at least a portion of a misfolded Aβ aggregate present; and
determining the presence of the soluble, misfolded Aβ protein in the sample by detecting at least a portion of the amplified portion of misfolded Aβ protein,
the soluble, misfolded Aβ protein comprising one or more of: a soluble, misfolded Aβ monomer and a soluble, misfolded Aβ aggregate; and
the amplified portion of misfolded Aβ protein comprising one or more of: an amplified portion of the soluble, misfolded Aβ monomer, an amplified portion of the soluble, misfolded Aβ aggregate, and an insoluble, misfolded Aβ aggregate.

2. The method of claim 1:
further comprising contacting an indicator of misfolded Aβ protein to the incubation mixture, the indicator of misfolded Aβ protein being characterized by an indicating state in the presence of misfolded Aβ protein and a non-indicating state in the absence of misfolded Aβ protein; and
wherein the determining the presence of the soluble, misfolded Aβ protein in the sample comprises detecting the indicating state of the indicator of misfolded Aβ protein in the incubation mixture.

3. The method of claim 2, the indicator of misfolded Aβ protein comprising one or more of: Thioflavin T, Congo Red, m-I-Stilbene, Chrysamine G, PIB, BF-227, X-34, TZDM, FDDNP, MeO-X-04, IMPY, NIAD-4, luminescent conjugated polythiophenes, a fluorescent protein, and derivatives thereof.

4. The method of claim 1, the determining the presence of the soluble, misfolded Aβ protein in the sample comprising:
determining an amount of the soluble, misfolded Aβ protein in the sample;
determining the amount of the soluble, misfolded Aβ protein in the sample at a sensitivity of at least about 80%;
determining the amount of the soluble, misfolded Aβ protein in the sample at less than about 100 nmol;
determining the amount of the soluble, misfolded Aβ protein in the sample in a molar ratio to monomeric, folded Aβ protein comprised by the sample, the molar ratio being less than about 1:100;
determining the soluble, misfolded Aβ protein in the sample with a specificity of at least about 80%; and
determining an amount of the soluble, misfolded Aβ protein in the sample compared to a control sample.

5. The method of claim 1, the incubating comprising incubating the incubation mixture at one or more of: between about 4° C. and about 35° C.

6. The method of claim 1, one or more of:
the detecting at least the portion of the amplified portion of misfolded Aβ protein comprising one or more of: a Western Blot assay, a dot blot assay, an enzyme-linked immunosorbent assay (ELISA), a thioflavin T binding assay, a Congo Red binding assay, a sedimentation assay, electron microscopy, atomic force microscopy, surface plasmon resonance, and spectroscopy; and
further comprising providing the monomeric, folded Aβ protein in labeled form, the detecting at least the portion of the amplified portion of misfolded Aβ protein comprising detecting the monomeric, folded Aβ protein in labeled form as incorporated into the amplified portion of misfolded Aβ protein.

7. The method of claim 1, the sample being taken from a subject, further comprising determining or diagnosing one or more of:
the presence of AD in the subject according to the presence of the soluble, misfolded Aβ protein in the sample;
the presence of AD in the subject according to the presence of the soluble, misfolded Aβ protein in the sample compared to a control sample taken from a control subject;
the presence of AD in the subject by comparing an amount of the soluble, misfolded Aβ protein in the sample to a predetermined threshold amount, the detecting at least the portion of the amplified portion of misfolded Aβ protein comprising detecting an amount of the soluble, misfolded Aβ protein in the sample;
the presence of AD in the subject according to the presence of the soluble, misfolded Aβ protein in the sample, the subject exhibiting no clinical signs of dementia according to cognitive testing;
the presence of AD in the subject according to the presence of the soluble, misfolded Aβ protein in the sample, the subject exhibiting no cortex plaques or tangles according to amyloid beta contrast imaging;
the presence of AD as a contributing factor to one or more clinical signs of dementia in the subject according to the presence of the soluble, misfolded Aβ protein in the sample, the subject exhibiting the one or more clinical signs of dementia according to cognitive testing; and
a progression or homeostasis of AD in the subject by comparing the amount of the soluble, misfolded Aβ protein in the sample to an amount of the soluble, misfolded Aβ protein in a comparison sample taken from the subject at a different time compared to the sample.

8. The method of claim 1, further comprising obtaining the sample from a subject,
the sample comprising one or more of: amniotic fluid; bile; blood; cerebrospinal fluid; cerumen; skin; exudate; feces; gastric fluid; lymph; milk; mucus; mucosal membrane; nasal secretions; peritoneal fluid; plasma; pleural fluid; pus; saliva; sebum; semen; sweat; synovial fluid; tears; and urine; and
the subject being one of a: human, mouse, rat, dog, cat, cattle, horse, deer, elk, sheep, goat, pig, and non-human primate.

9. The method of claim 1, the subject being treated with an Aβ modulating therapy, further comprising:
comparing the amount of the soluble, misfolded Aβ protein in the sample to an amount of the soluble, misfolded Aβ protein in a comparison sample, the sample and the comparison sample being taken from the subject at different times over a period of time under the Aβ modulating therapy; and
determining the subject is one of: responsive to the Aβ modulating therapy according to a change in the soluble, misfolded Aβ protein over the period of time, or non-responsive to the Aβ modulating therapy according to homeostasis of the soluble, misfolded Aβ protein over the period of time.

10. The method of claim 1, the sample being taken from a subject, the subject being treated with an Aβ modulating therapy, the Aβ modulating therapy comprising administration of one or more of: an inhibitor of BACE1 (beta-secretase 1); an inhibitor of γ-secretase; a modulator of Aβ homeostasis; E2609; MK-8931; LY2886721; AZD3293; semagacestat (LY-450139); avagacestat (BMS-708163); solanezumab; crenezumab; bapineuzumab; BIIB037; CAD106; antibodies raised against Aβ globulomers; ACC-001; V950; and Affitrope AD02.

11. The method of claim 1, the monomeric, folded Aβ protein and/or the soluble, misfolded Aβ protein comprising one or more of: peptides formed via β- or γ-secretase cleavage of amyloid precursor protein; Abeta40; and Abeta42.

12. The method of claim 1, comprising:
contacting the sample with Thioflavin T and the monomeric, folded Aβ protein to form the incubation mixture;
conducting the incubation cycle two or more times on the incubation mixture effective to form the amplified portion of misfolded Aβ protein from the monomeric, folded Aβ protein, each incubation cycle comprising:
incubating the incubation mixture effective to cause misfolding and/or aggregation of at least the portion of the monomeric, folded Aβ protein in the presence of the soluble, misfolded Aβ protein;
shaking the incubation mixture effective to at least partly de-aggregate at least the portion of a misfolded Aβ aggregate present; and
determining the presence of the soluble, misfolded Aβ protein in the sample by detecting a fluorescence of the Thioflavin T corresponding to at least the portion of the amplified portion of misfolded Aβ protein.

13. A method for determining a presence of a soluble, misfolded Aβ protein in a sample, comprising:
capturing a soluble, misfolded Aβ protein from the sample to form a captured soluble, misfolded Aβ protein;
contacting the captured, soluble misfolded Aβ protein with a molar excess of monomeric, folded Aβ protein to form an incubation mixture, the molar excess being greater than an amount of Aβ protein monomer included in the captured soluble, misfolded Aβ protein;
conducting an incubation cycle two or more times on the incubation mixture effective to form an amplified portion of misfolded Aβ protein from the monomeric, folded Aβ protein, each incubation cycle comprising:
incubating the incubation mixture effective to cause misfolding and/or aggregation of at least a portion of the monomeric, folded Aβ protein in the presence of the captured soluble, misfolded Aβ protein;
physically disrupting the incubation mixture effective to at least partly de-aggregate at least a portion of a misfolded Aβ aggregate present; and
determining the presence of the soluble, misfolded Aβ protein in the sample by detecting at least a portion of the amplified portion of misfolded Aβ protein,
the soluble, misfolded Aβ protein comprising one or more of: a soluble, misfolded Aβ monomer and a soluble, misfolded Aβ aggregate; and
the captured, soluble, misfolded Aβ protein comprising one or more of: a captured, soluble, misfolded Aβ monomer and a captured, soluble, misfolded Aβ aggregate; and
the amplified portion of misfolded Aβ protein comprising one or more of: an amplified portion of the soluble, misfolded Aβ monomer, an amplified portion of the soluble, misfolded Aβ aggregate, and an insoluble, misfolded Aβ aggregate.

14. The method of claim 13, the capturing comprising selectively concentrating the soluble, misfolded Aβ protein in one or more of the sample and the incubation mixture.

15. The method of claim 14, the selectively concentrating the soluble, misfolded Aβ protein comprising one or more of: pre-treating the sample prior to forming the incubation mixture and pre-treating the incubation mixture prior to incubating the incubation mixture.

16. The method of claim 14, the selectively concentrating the soluble, misfolded Aβ protein comprising contacting one or more Aβ specific antibodies to the soluble, misfolded Aβ protein to form a captured soluble, misfolded Aβ protein, the one or more Aβ specific antibodies comprising one or more of: 6E10, 4G8, 82E1, A11, X-40/42, 16ADV, an antibody specific for an amino acid sequence of Aβ, and an antibody specific for a conformation of the soluble, misfolded Aβ protein.

17. The method of claim 16, the one or more Aβ specific antibodies being coupled to a solid phase.

18. The method of claim 17, the solid phase comprising one or more of a magnetic bead and a multiwell plate.

19. The method of claim 13, the subject being treated with an Aβ modulating therapy, further comprising:
comparing the amount of the soluble, misfolded Aβ protein in the sample to an amount of the soluble, misfolded Aβ protein in a comparison sample, the sample and the comparison sample being taken from the subject at different times over a period of time under the Aβ modulating therapy; and
determining the subject is one of: responsive to the Aβ modulating therapy according to a change in the soluble, misfolded Aβ protein over the period of time, or non-responsive to the Aβ modulating therapy according to homeostasis of the soluble, misfolded Aβ protein over the period of time.

20. The method of claim 13, the sample being taken from a subject, further comprising determining or diagnosing one or more of:
the presence of AD in the subject according to the presence of the soluble, misfolded Aβ protein in the sample;
the presence of AD in the subject according to the presence of the soluble, misfolded Aβ protein in the sample compared to a control sample taken from a control subject;
the presence of AD in the subject by comparing an amount of the soluble, misfolded Aβ protein in the sample to a predetermined threshold amount, the detecting at least the portion of the amplified portion of misfolded Aβ protein comprising detecting an amount of the soluble, misfolded Aβ protein in the sample;
the presence of AD in the subject according to the presence of the soluble, misfolded Aβ protein in the sample, the subject exhibiting no clinical signs of dementia according to cognitive testing;
the presence of AD in the subject according to the presence of the soluble, misfolded Aβ protein in the sample, the subject exhibiting no cortex plaques or tangles according to amyloid beta contrast imaging;
the presence of AD as a contributing factor to one or more clinical signs of dementia in the subject according to the presence of the soluble, misfolded Aβ protein in the sample, the subject exhibiting the one or more clinical signs of dementia according to cognitive testing; and
a progression or homeostasis of AD in the subject by comparing the amount of the soluble, misfolded Aβ protein in the sample to an amount of the soluble, misfolded Aβ protein in a comparison sample taken from the subject at a different time compared to the sample.
